(19) United States
(12) Patent Application Publication
(10) Pub. No.: US 2005/0113689 A1
(43) Pub. Date: May 26, 2005

(54) Method and apparatus for performing multi-mode imaging

(76) Inventor: Arthur Gritzky, Pollham (AT)

Correspondence Address: Dean D. Small;Armstrong Teasdale LLP, Suite 2600, One Metropolitan Square, St. Louis, MO, 63102, US

(21) Appl. No.: 10/860,188

(22) Filed: Jun. 2, 2004

Related U.S. Application Data
(60) Provisional application No. 60/524,323, filed on Nov. 21, 2003.

(51) Int. Cl. A61B 8/00; A61B 8/14

(52) U.S. Cl. 600/437; 600/440

(57) ABSTRACT

A method and apparatus for performing multi-mode imaging are provided. The includes performing a first volume scan using a first mode to acquire a first data set and performing a second scan using a second mode to acquire a second data set. The first and second modes are different.

### CROSS REFERENCE TO RELATED APPLICATIONS

This application claims priority to and the benefit of the filing date of U.S. Provisional Application No. 60/524,323, filed on Nov. 21, 2003 and which is hereby incorporated by reference in its entirety.

### BACKGROUND OF THE INVENTION

This invention relates generally to diagnostic ultrasound systems. In particular, the present invention relates to methods and devices for acquiring multiple images with diagnostic ultrasound systems using different modes of operation.

A variety of known ultrasonic transducers are used to acquire diagnostic image data. Often, a transducer that is designed for one or several types of applications, or modes of operation, is unable to function to provide other desirable modes of operation. Further, other known transducers may be capable of operating in multiple modes, but are limited to conventional two-dimensional (2D) scanning. For example, conventional 2D transducers may intersperse beams to acquire a first type of data, such as B-mode data, with beams to acquire a second type of data, such as M-mode and PW Doppler data. However, data acquired with different modes to provide different functionality and/or views of the same anatomy is limited to 2D data. Thus, in many circumstances (e.g., when not using 2D data), data must be acquired and saved in a first mode, then the transducer must be switched to a different mode, with data then acquired and saved in that second mode. This may result in the need for rescanning a patient and longer examination times.

Thus, these known devices are limited in their ability to operate in multiple modes to acquire multi-mode data sets while providing different scanning options, for example, when using a volume transducer.

### BRIEF DESCRIPTION OF THE INVENTION

In one exemplary embodiment, a method for performing multi-mode ultrasonic imaging is provided. The method includes performing a first volume scan using a first mode to acquire a first data set and performing a second scan using a second mode to acquire a second data set. The first and second modes are different.

In another exemplary embodiment, a method of performing a multi-mode ultrasonic acquisition of an object is provided. The method includes acquiring a first data set containing at least two dimensions of spatial information and one dimension of at least one of temporal and spatial information. The method further includes acquiring a second data set separate from and simultaneously with said first data set. The second data set contains at least a first dimension containing spatial information and a second dimension containing one of spatial, motion and temporal information.

In yet another exemplary embodiment, an ultrasound system is provided that includes a probe for acquiring a first data set with a volume scan in a first mode and acquiring a second data set with a scan in a second mode. The first and second modes are different. The ultrasound system further includes a processor configured to process the first and second data sets for display as first and second images, with the first and second images displayed on the same display.

### BRIEF DESCRIPTION OF THE DRAWINGS

FIG. 1 illustrates a block diagram of an ultrasound system formed in accordance with an embodiment of the present invention.

FIG. 2 illustrates an ultrasound system formed in accordance with another embodiment of the present invention.

FIG. 3 illustrates a scan sequence in a first direction in accordance with an embodiment of the present invention.

FIG. 4 illustrates a scan sequence in a second direction in accordance with an embodiment of the present invention.

FIG. 5 illustrates two images displayed in real-time in accordance with an embodiment of the present invention.

FIG. 6 illustrates an example of filtering that may be used to correct movement artifacts in accordance with an embodiment of the present invention.

FIG. 7 illustrates first and second images displayed in real-time in accordance with an embodiment of the present invention.

FIG. 8 illustrates first, second, and Nth images displayed in accordance with an embodiment of the present invention.

FIG. 9 illustrates first and second images displayed in real-time in accordance with an embodiment of the present invention.

FIGS. 10-13 illustrates examples of probes that may be used to acquire scan data in multiple modes in real-time in accordance with an embodiment of the present invention.

FIG. 14 illustrates a volumetric scan acquired in accordance with an embodiment of the present invention.

### DETAILED DESCRIPTION OF THE INVENTION

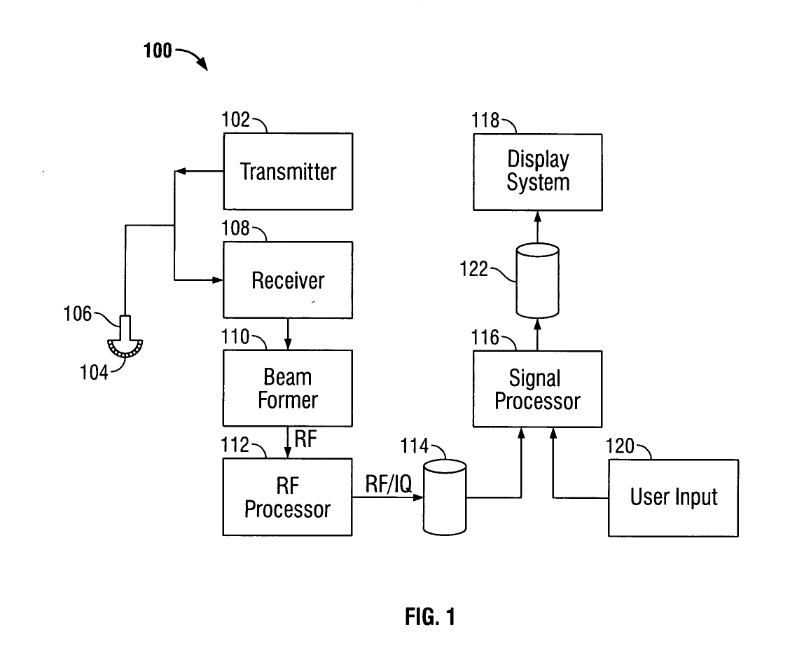

FIG. 1 illustrates a block diagram of an ultrasound system 100 formed in accordance with an embodiment of the present invention. The ultrasound system 100 includes a transmitter 102 which drives an array of elements 104 within a volume transducer 106 to emit pulsed ultrasonic signals into a body. The volume transducer 106 may be a mechanical transducer, a 2D array transducer, and the like. A variety of geometries may be used. Further, the volume transducer 106 may be provided, for example, as part of a volume probe (not shown). The ultrasonic signals are back-scattered from structures in the body, like blood cells or muscular tissue, to produce echoes which return to the elements 104. The echoes are received by a receiver 108. The received echoes are communicated to a beamformer 110, which performs beamforming and outputs an RF signal. The RF signal then are provided to an RF processor 112. Alternatively, the RF processor 112 may include a complex demodulator (not shown) that demodulates the RF signal to form IQ data pairs representative of the echo signals. The RF or IQ signal data may then be routed directly to an RF/IQ buffer 114 for temporary storage. A user input 120 may be used to input patient data, scan parameters, a change of scan mode, and the like.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation.

The ultrasound system 100 may continuously acquire volumetric ultrasound information at a frame rate that exceeds, by way of example only, twenty volumes per second. The acquired ultrasound information may be displayed on the display system 118 at a slower frame rate. An image buffer 122 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

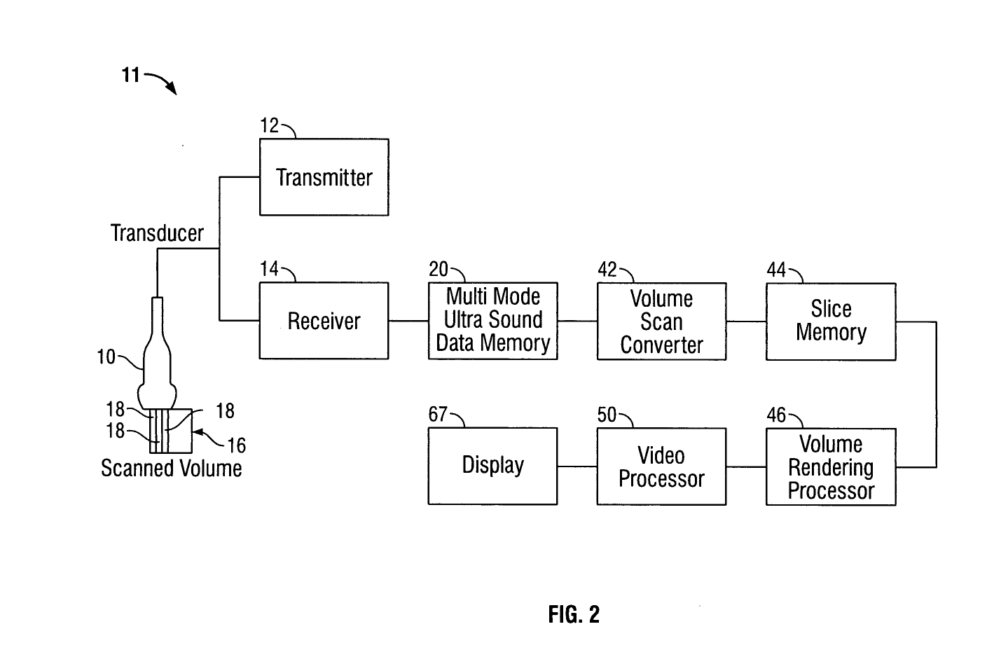

FIG. 2 illustrates an ultrasound system 11 formed in accordance with another embodiment of the present invention. The system 11 includes a transducer 10 connected to a transmitter 12 and a receiver 14. The transducer 10 transmits ultrasonic pulses and receives echoes from structures inside of a scanned ultrasound volume 16. A multiple mode ultrasound data memory 20 stores ultrasound data from the receiver 14 derived from the scanned ultrasound volume 16. The volume 16 may be obtained by various techniques (e.g., 3D scanning, real-time 3D imaging, volume scanning, 2D or matrix array transducers and the like).

The volume 16 may be acquired by a volumetric transducer, such as a mechanical or 2D array (e.g., electrically steerable) transducer 10. Scan planes 18 or volume 16 are stored in the multiple mode ultrasound data memory 20, and then provided to a volume scan converter 42. In some embodiments, the transducer 10 may obtain lines instead of the scan planes 18, and the memory 20 may store lines obtained by the transducer 10 rather than the scan planes 18. The volume scan converter 42 creates a data slice from multiple adjacent scan planes 18. The data slice is stored in slice memory 44 and is accessed by a volume rendering processor 46. The volume rendering processor 46 performs volume rendering upon the data slice. The output of the volume rendering processor 46 is provided to the video processor 50 and the displayed on display 67. The volume rendering processor 46 is adapted to perform one or more processing operations according to a plurality of selectable ultrasound modalities on the acquired ultrasound information.

It should be noted that the position of each echo signal sample (Voxel) is defined in terms of geometrical accuracy (i.e., the distance from one Voxel to the next) and ultrasonic response (and derived values from the ultrasonic response). Suitable ultrasonic responses include, for example, gray scale values, color flow values, and angio or power Doppler information.

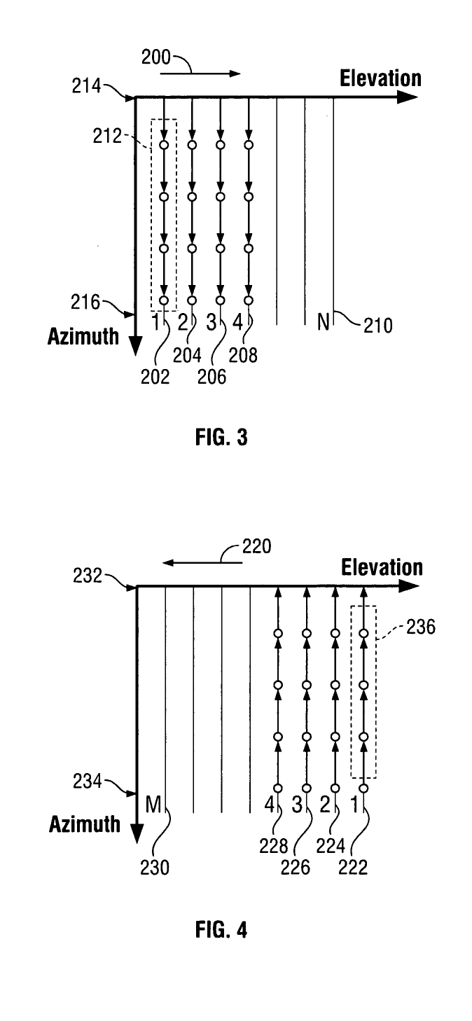

FIG. 3 illustrates an exemplary embodiment a scan sequence in a first direction acquired, for example, by a mechanical transducer 10. The transmitter 12 transmits ultrasound pulses or firings and collects ultrasound echo data based on a first mode of operation. The first mode of operation may be, for example, 2D, 3D or 4D, and/or may be one of B-mode, Power Doppler, Pulse Wave Doppler, Continuous Wave Doppler, Color Doppler, Harmonic Imaging, and Color Flow, and the like. The mode of operation may be selected or predefined by the user. In FIG. 3, the first direction 200 is illustrated as the positive elevation direction. A mechanical transducer 10, such as transducers with internal rotating elements, wobbling transducers and annular phased arrays may be used. In addition, phased array, linear array, curved and convex array, and sector transducers 10 may be used. A general discussion concerning transducers 10 is included below. For example, to acquire 3D or 4D data with color, a volumetric transducer 10 having a defined geometry or a 2D array may be used.

FIG. 14 illustrates a volumetric scan 400 in accordance with an exemplary embodiment of the present invention. The volumetric transducer 10 (shown in FIG. 2) performs a first volume scan 406 using a first mode of operation, such as B-mode, over a scan geometry 402, for example, a predetermined scan range of sixty degrees. This may be performed, for example, using any suitable mechanical or electrical means for scanning. The volumetric transducer 10 then performs a second volume scan 408 using a second mode of operation, such as Power Doppler, and a different geometry, such as a second scan range 404, for example, thirty degrees. The second volume scan 408 may also be a slice or line of data. It should be understood that the modes of operation are not limited to the above, and also may include 2D, 3D and 4D, and B-mode, Power Doppler, Pulse Wave Doppler, Continuous Wave Doppler, Harmonic Imaging, B-flow, and the like. In addition, the second mode of operation may be accomplished at a different volume scanning rate or speed compared to the first mode of operation. The first and second volume scans 406 and 408 also may be acquired at different positions in space by focusing the elements 104 (shown in FIG. 1) and without movement of the volumetric transducer 10, such as with a 2D array transducer 10. In addition, the first and second volume scans 406 and 408 may be acquired at a faster scan rate with respect to scans acquired with a mechanical transducer 10 as the 2D array transducer 10 does not require movement to a second or Nth position.

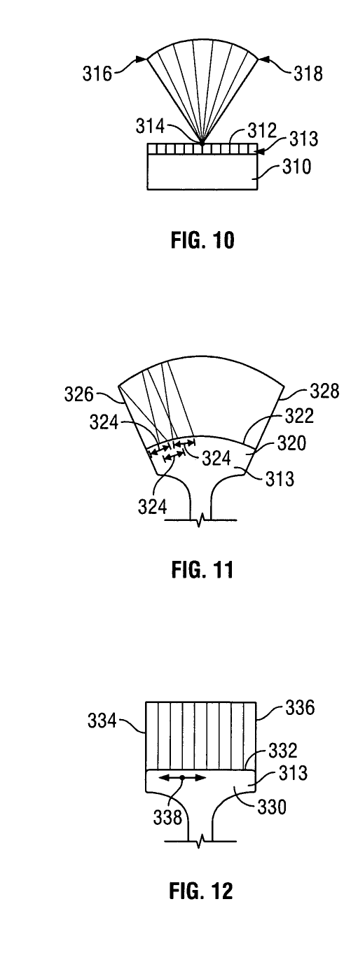

Returning to FIG. 3, the transducer 10 scans, or is moved, in a first direction 200. The transducer 10 may scan continuously or move in predefined increments. Alternatively, the focus of the elements 104 (shown in FIG. 1) may move in the first direction 200. Therefore, a patient is scanned sequentially from scan planes 1 202 through scan plane N 210. In an exemplary embodiment, a scan sequence 212 is received starting at a top 214, or patient surface, and moving to a bottom 216 of the scan planes 202-210. In other words, when acquiring data from scan planes 202-210, the transducer 10 receives echoes returned from tissue closest to the transducer 10 first, and echoes returned from tissue furthest from the transducer 10 last for each scan plane 202-210. The scan sequence 212 is repeated for each scan plane 202-210 consecutively. However, it should be noted that non-sequential scanning also may be provided. Although the scan planes 202-210 are illustrated as perpendicular lines, it should be understood that when acquiring data using different types of transducers 10, such as mechanical transducers 10, the scan planes 202-210 may be represented by a slight tilt. Other transducers 10 also may create differently positioned scan planes 202-210, such as a sector, which may be acquired by a virtual convex transducer 340 (shown in FIG. 10). The volume rendering processor 46 (shown in FIG. 2) processes the received echo data to create a first data set. For example, data representative of slices of a scan may be combined for display. A first image is then displayed on the display 67 (shown in FIG. 2) based on the first data set.

When using a non-mechanical transducer 10 (e.g., electrically steerable), beamforming during transmit and receive operation may be performed such that the scan sequences may be modified based on, for example, the type of scan. The sequencing and activation of the individual elements of the transducer 10 may be controlled such that, for example, the scan beam may be tilted (e.g., between ten degrees and twenty degrees) using electrical steering. Further, an interleaved scan or an additive scan may be performed. During an interleaved scan, a portion of a scan for a first volume is performed, followed by a portion of a scan of a second volume, followed by another portion of a scan of the first volume, which process continues until both volumes are scanned (e.g., ten slices of a first volume, five slices of a second volume, ten slices of a first volume, etc.). During an additive scan, a first volume is scanned, then a second volume is scanned and the scans combined.

It should be noted that the various embodiments of the invention described herein are not limited to the ultrasound systems 11 and 100, but may be used with other ultrasound systems. Further, although certain embodiments are described in connection with one of the ultrasound systems 11 or 100 using component parts of that ultrasound system, it is not so limited, and the embodiments may be implemented in connection with the other ultrasound system. For example, the volume transducer 106 (shown in FIG. 1) with elements 104 may be operated (e.g., mechanically or electrically) to acquire echo data, which is the processed by the signal processor 116 (shown in FIG. 1) to create a data set (e.g., the first data set).

FIG. 4 illustrates an exemplary embodiment of a scan sequence in a second direction acquired, for example, by a mechanical transducer 10. The transducer 10 (shown in FIG. 2), or the focus of the elements 104 (shown in FIG. 1), is moved in a second direction 220 as discussed previously. In FIG. 4, the second direction 220 is illustrated as the negative elevation or opposite direction to the first direction 200. However, the second direction 220 is not limited to the negative elevation direction of the first direction 220 and may, for example, be provided at an angle relative to the first direction 200. The transmitter 12 (shown in FIG. 2) transmits ultrasound firings and receives echoes based on a second mode of operation. The second mode of operation is different from the first mode, and may be any one of the modes listed previously. In addition, modes such as anatomical M-mode, M-mode, and modes used to display one or more slices of data based on the first image also may be used.

Echo data is received sequentially from scan plane 1 222 through scan plane M 230. However, it should be noted that non-sequential scanning also may be provided. In an exemplary embodiment, a scan sequence 236 is received starting at a bottom 234, furthest from the surface of the transducer 10, and moving to a top 232 of the scan planes 222-230. The processor 116 (shown in FIG. 1) processes the received echo data as described herein to create a second data set. A second image is then displayed on the display 67 based on the second data set. It should be noted that the number of N and M scan planes do not need to be equal.

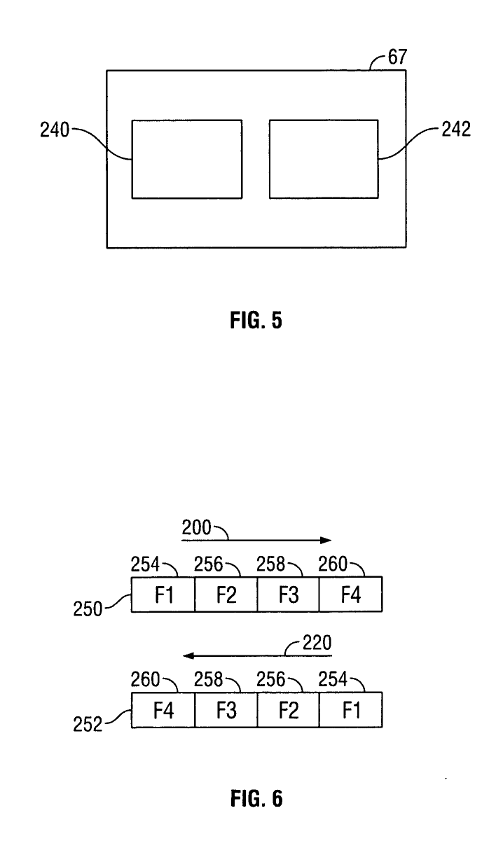

FIG. 5 illustrates two images displayed on display 67 in real-time. A first image 240 is displayed based on the first data set received from scanning in the first direction 200, such as in FIG. 3. A second image 242 is displayed based on the second data set received from scanning in the second direction 220, such as in FIG. 4. The first and second images 240 and 242 are acquired using different modes, but are based on the same anatomy, and thus present diagnostic data in different ways (e.g., forms from different modes of operation) to the user which may be contrasted and compared in real-time. Additionally, the first image 240, such as a B-mode volume, may be used to orient the user with respect to the second image 242. Alternatively, the first image 240 may be displayed based on the first volumetric scan 406 (shown in FIG. 14) and the second image 242 may be displayed based on the second volumetric scan 408 (shown in FIG. 14).

By way of example only, when scanning in the first direction 200, the transducer 10 may transmit and receive data to create the first image 240 as a 4D B-mode image. When scanning in the second direction 220, the transducer 10 transmits and receives data to create the second image 242. For Doppler and Color modes, the transducer 10 transmits at least two firings along the same plane 222-230. Acquiring Doppler and Color will increase the amount of data, and the frame rate may be cut in half. Therefore, the ultrasound system 11 or 100 may utilize multi-line acquisition, wherein the transducer 10 receives at least two pulses from different spatial locations for every transmission. Additionally, multi-transmit and multi-receive may be used when acquiring 4D color flow.

The first and second images 240 and 242 may be acquired, for example, using different sampling rates, resolutions, and/or different frame rates. For example, when using mechanical transducers, multiple transmit/multiple receive may be used to increase the frame rate in one or both of the first and second directions 200 and 220. The scan speeds may be varied to acquire the first and second data sets. By way of example only, B-mode volume data may be acquired in the first direction 200 at a slow scan speed to acquire more data, while color data may be acquired in the second direction 220 at a higher scan speed.

In an exemplary embodiment, with transducers 10 utilizing electronic 2D arrays, resolution may be varied by scanning different amounts of data. For example, scanning a one degree sector of data results in a higher resolution compared to scanning a three degree sector of data. When acquiring B-mode, the transmit and receive beams are closer together than beams when acquiring color data. The color data resolution may be increased by using a multiple transmit/multiple receive technique. In addition, two different modes may acquire scan information at different frame rates. By way of example only, a 4D B-mode volume may be acquired at a lower rate while Pulse Wave Doppler may be acquired at a higher rate, a 4D B-mode volume may be acquired at a lower rate while an anatomic M-mode may be acquired at a higher rate, and/or a 4D B-mode volume having low resolution may have a slower update rate while a higher resolution 2D slice may have a higher update rate. Thus, different planes having different resolutions may be updated at different rates. In an exemplary embodiment, a user may modify frame rates, update rates, and/or control the mode of operation with a user control, for example, the user input 120.

Movement of the transducer 10 and/or the tissue of the patient may result in movement artifacts, such as background noise, speckle, clutter (associated with color), and smearing background color. For example, moving the transducer 10 on the patient surface creates a spatial shift in the received scan data. Scan data is received later in time, so the time needs to be corrected. Therefore, the global movement of the scan lines of planes must be estimated and corrected.

FIG. 6 illustrates an exemplary embodiment of filtering that may be used to correct movement artifacts. The processor 116 uses a first filter 250 to filter the first data set acquired in the first direction 200, and a second filter 252 to filter the second data set acquired in the second direction 220. Examples of temporal filters that may be used include filters that provide averaging using the radial distance from the transducer 10 as a reference, wherein two or more scan lines 202-210 and 222-230 are averaged together, Gaussian kernel, convolution kernel, and the like. Additionally, the first filter 250 is illustrated having filter values F1 254-F4 260, which the processor 116 applies consecutively to filter the first data set acquired in the first direction 200. For the second direction 220, the processor 116 reverses the first filter 250 in time to create the second filter 252. The processor 116 then filters the second data set with the second filter 252.

In another embodiment, a finite impulse response (FIR) filter having coefficients may be used. The first filter 250 comprises coefficients. The implementation of the first filter 250 is reversed, such as by mirroring coefficients, to create the second filter 252.

The first and second filters 250 and 252 may be applied on a pixel by pixel basis, a scan line by scan line basis, or to subsets of scan lines. In addition, global clutter filtering may be used to estimate global movement of each scan plane 202-210 and 222-230. It should be noted that additional weighting within the filter or kernel may be added and/or adjusted to compensate for the shifting of data due to the scanning motion.

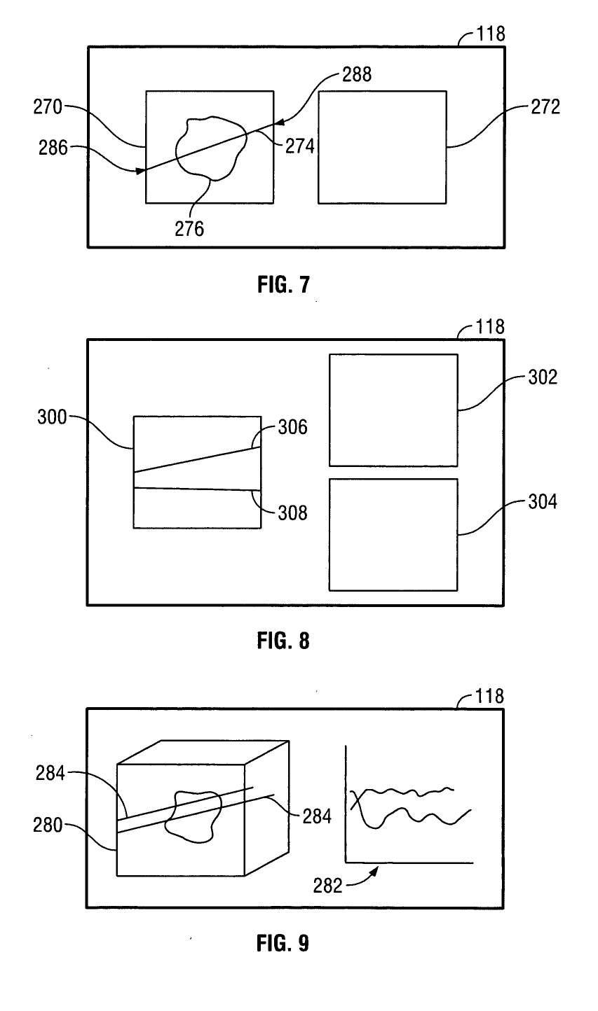

Thus, as shown in FIG. 7, first and second images 270 and 272 may be displayed on display system 118 in real-time. The user may use a user control, such as, for example, the user input 120 (shown in FIG. 1) to select, for example, a protocol defining the modes of operation, and thus the type of images 270 and 272. It should be noted that the user may use the user input 120 to select and/or change the modes of operation or types of images 270-272. Further, it should be noted that the processor 116 (shown in FIG. 1) or processor 46 (shown in FIG. 2) may automatically define a filter 250 and 252 to be used or may prompt the user to select or input the type of filter 250 and 252.

In the exemplary embodiment shown in FIG. 7, the first image 270 has been defined or selected to be a 3D or 4D B-mode volume and the second image 272 is a slice of data based on the first image 270. The transducer 106 (shown in FIG. 1) acquires the scan data in the first direction 200. The processor 116 (shown in FIG. 1) processes and filters the scan data with the first filter 250 as described herein and creates the first data set. The display system 118 then displays and/or updates in real-time the first image 270 comprising the anatomy of interest 276 based on the first data set. It should be noted that the first image 270 may be displayed in a larger format while it is the only image being displayed. Also, the transducer 10 may acquire additional scan data in the second direction 220 using the first mode of operation, and use the additional scan data to update the first image 270.

The user may define at least one slice of interest 274 through the anatomy of interest 276 on the first image 270 with the user input 120 (shown in FIG. 1). The slice of interest 274 may comprise, for example, a C-plane slice of data. The transducer 106 also acquires scan data in the second direction 220 using the second mode of operation. The processor 116 filters the scan data with the second filter 252 as described herein and creates the second data set. The display system 118 then displays and/or updates the second image 272 based on the slice of interest 274.

The update rate of the second image 272 may be limited by the time required to scan from a beginning 286 to an end 288 of the slice of interest, or by the orientation of the slice of interest 274 with respect to the scan planes 202-210 (shown in FIG. 3). By way of example only, the first image 270 (e.g., 4D B-mode volume) may be updated at a rate of two volumes per second, while the second image 272 may be updated at ten to fifteen volumes per second.

Alternatively, the slice of interest 274 may be displayed automatically in an arbitrary position on the first image 270, and the second image 272 is displayed based on the arbitrary position of the slice of interest 274. The user then may rotate and change the location, thickness, and the like, of the slice of interest 274 with user input 120. It should be noted that scan data outside the slice of interest 274 may be disregarded so that the data is not saved or processed.

FIG. 8 illustrates first, second, and Nth images 300-304 on display system 118. The user may define multiple slices of interest 306 and 308 to create additional images. The first data set is acquired and processed as previously discussed. The transducer 106 acquires data in the second direction 220, and the processor 116 filters the data with the second filter 252 and further processes the data to create two data sets. Each data set is used to display an image, such as second and Nth images 302 and 304. The slices of interest 306 and 308 may be defined, for example, to have different resolutions and/or different update rates. It should be understood that although FIG. 8 displays three images, additional images based on additional slices of interest (not shown) may be defined and displayed. Further, the user may define the second and Nth images 302 and 304 based on a single slice of interest 306 and 308. The user may define the second and Nth images 302 and 304 to have, for example, different resolutions, different update rates, and the like as previously discussed.

FIG. 9 illustrates first and second images 280 and 282 on the display system 118 in real-time. The first image 280 in FIG. 9 illustrates a 4D B-mode volume. The anatomy being scanned may be, for example, a heart, heart valve, artery, vein, and the like.

The second image 282 illustrates an anatomic M-mode scan based on one or more lines 284 defined on the first image 280. As previously discussed, the lines 284 may be defined by the user once the first image 280 is displayed, or may be automatically displayed and then moved to the desired location by the user.

As the lines 284 are defined on the first image 280, the processor 116 (shown in FIG. 1) filters the data acquired in the second direction 220 and creates the second data set. The second image 282 then displays the M-mode data based on the one or more lines 284. First and second images 280 and 282 are acquired at different frame rates, wherein the second image 282 comprising the M-mode data is at a higher frame rate.

FIGS. 10-13 illustrate examples of transducers 10 or 106 that may be used to acquire scan data in multiple modes in real-time. It should be understood that the illustrated transducers 10 and 106, and other transducers not illustrated or specified, but known, may be used to implement the various embodiments of the present invention described herein, including the various acquisition techniques.

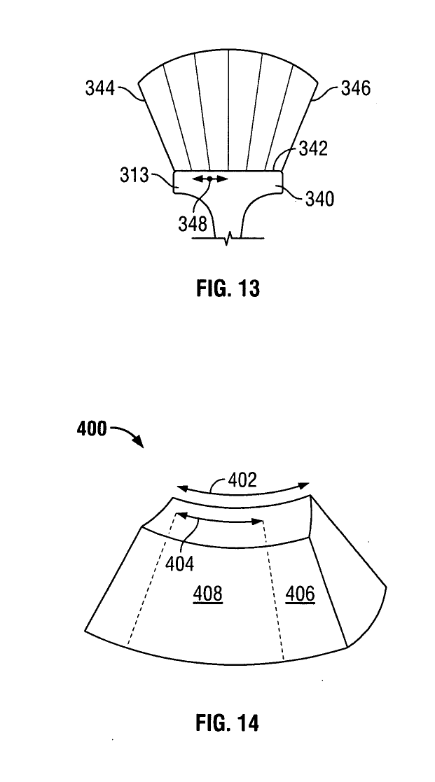

For example, as shown in FIG. 10, a phased array transducer 310 comprises a linear surface 312 having a small aperture. Ultrasonic beams originate in about a middle 314 of the 2D array 313 of elements. Firings (e.g., emitted pulsed ultrasonic signals) of the 2D array 313 are timed to steer the focus back and forth between first and second sides 316 and 318. As shown in FIG. 11, a curved array transducer 320 comprises a curved surface 322 and has a 2D array 313 of elements. Subsets 324 of the array 313 may be fired at different times as the scan moves between first and second sides 326 and 328. As shown in FIG. 12, a linear array transducer 330 with a linear surface 332 has a 2D array 313 of elements and scans between first and second sides 334 and 336. The linear array 330 has an origin or center 338 that moves as the scan is acquired. As shown in FIG. 13, a virtual convex transducer 340 with a linear surface 342 has and has a 2D array 313 of elements and scans between first and second sides 344 and 346. The elements are steered to form a sector scan as the center 348 moves.

While the invention has been described in terms of various specific embodiments, those skilled in the art will recognize that the invention can be practiced with modification within the spirit and scope of the claims.

1. A method for performing multi-mode ultrasonic imaging comprising:
performing a first volume scan using a first mode to acquire a first data set; and
performing a second scan using a second mode to acquire a second data set, the first and second modes being different.

2. A method in accordance with claim 1, wherein the second scan comprises one of a plane and single line scan.

3. A method in accordance with claim 1, wherein the second scan comprises a volume scan.

4. A method in accordance with claim 3, further comprising:
acquiring the first data set at a first volume rate; and
acquiring the second data set at a second volume rate, the first and second volume rates being different.

5. A method in accordance with claim 1, wherein the first and second modes comprise at least one of b-mode, Power Doppler, Pulse Wave Doppler, Continuous Wave Doppler, Harmonic Imaging and color flow imaging.

6. A method in accordance with claim 1, further comprising:
displaying a first image based on the first data set; and
displaying a second image based on the second data set, the first and second images displayed at the same time.

7. A method in accordance with claim 6, wherein the first and second images are displayed on a single display in real-time.

8. A method in accordance with claim 1, further comprising:
displaying a first image based on a volume frame rate of the first data set; and
identifying a portion on the first image with the second data set acquired based on the identified portion and a second volume frame rate.

9. A method in accordance with claim 8, wherein the portion comprises at least one of a volume, slice, line and plane.

10. A method in accordance with claim 8, wherein the identifying comprises identifying a plurality of portions.

11. A method in accordance with claim 10, further comprising displaying a plurality of images corresponding to the plurality of identified portions.

12. A method in accordance with claim 10, wherein the plurality of portions are defined having at least one of different resolutions and update rates.

13. A method in accordance with claim 1, wherein performing a first volume scan comprises scanning in a first direction in the first mode to acquire the first data set and performing a second scan comprises scanning in a second direction in the second mode to acquire the second data set.

14. A method in accordance with claim 13, wherein the first and second directions are opposite.

15. A method in accordance with claim 1, further comprising selecting the first and second modes of operation based upon a user input.

16. A method in accordance with claim 1, further comprising filtering the first and second data sets.

17. A method in accordance with claim 1, wherein the scans are performed using one of a probe having a mechanically controlled array and a probe having an electrically controlled array.

18. A method is accordance with claim 1, wherein the first volume scan and the second volume scan are performed using an interleaved operation.

19. A method is accordance with claim 1, wherein the first volume scan and the second volume scan are performed during a single scanning operation.

20. A method of performing a multi-mode ultrasonic acquisition of an object, comprising:
acquiring a first data set containing at least two dimensions of spatial information and one dimension of at least one of temporal and spatial information; and
acquiring a second data set separate from and simultaneously with said first data set, said second data set containing at least a first dimension containing spatial information and a second dimension containing one of spatial, motion and temporal information.

21. An ultrasound system comprising:
a probe for acquiring a first data set with a volume scan in a first mode and acquiring a second data set with a scan in a second mode, the first and second modes being different; and
a processor configured to process the first and second data sets for display as first and second images, the first and second images displayed on the same display.

22. An ultrasound system in accordance with claim 21, wherein the probe is configured to scan in a first direction to acquire the first data set and to scan in a second direction to acquire the second data set, the first and second directions being different.
